(19) United States
(12) Patent Application Publication
(10) Pub. No.: US 2026/0234174 A1
(43) Pub. Date: Aug. 13, 2026

(54) PHOTOBASES

(71) Applicant: Technische Universität Wien, Wien (AT)

(72) Inventors: Klaus ABLEIDINGER, Wien (AT); Robert LISKA, Wien (AT); Jurgen STAMPFL, Wien (AT)

(21) Appl. No.: 19/160,324

(22) PCT Filed: Feb. 27, 2024
(86) PCT No.: PCT/EP2024/055004

§ 371 Date: Aug. 27, 2025

(30) Foreign Application Priority Data
Feb. 28, 2023 (AT) A 60031/2023
Aug. 7, 2023 (AT) A 93/2023

(51) Int. Cl. C07F 9/54; C07C 59/205; C07D 311/86; C07D 335/16; C09D 7/63

(52) U.S. Cl. CPC C07F 9/5442; C07C 59/205; C07D 311/86; C07D 335/16; C09D 7/63

(57) ABSTRACT

A triphenylphosphonium carboxylate salt of the following Formula (I) may be used as a photobase generator in a photopolymerizable composition containing at least one type of monomer for preparing photopolymers via curing the composition by irradiating with light of a suitable wavelength:

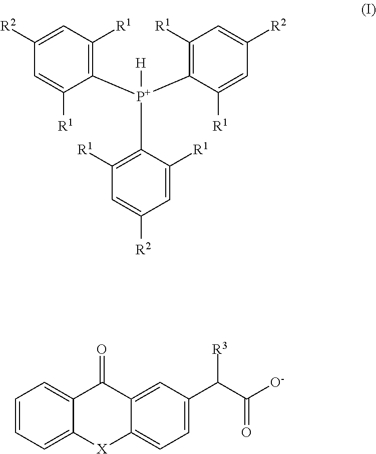

R¹ and R² are each independently selected from —H and —OCH₃, on each of the three phenyl radicals at least one of R¹ and R² represents —OCH₃, and R³ is selected from —H or —CH₃ and X is either absent or selected from a chemical bond, —CH₂—, —O—CH₂—, —CH₂—O—, —C(═O)—, —O— or —S—.

The present invention relates to the use of novel carboxylate salts as photobase generators in photopolymerizable compositions.

### STATE OF THE ART

The concept of organic photobase generators (PBG) was first introduced in 1990 by Cameron and Frechet who used a photolabile carbamate group for preparing basic amines (J. F. Cameron, J. M. J. Frechet, J. Org. Chem. 55, 5919-5922 (1990)). Later reports describe the light-induced release of primary, secondary or tertiary amines, which are rather weak bases and thus inefficient for the activation of anionic polymerization. The use of salts for preparing bases was first described in a report from 1998 in the form of quaternary ammonium salts (Sarker et al., J. Phys. Chem. A 102, 5375-5382 (1998)), which strongly increased the storage stability of the formulations. In recent years, numerous carboxylates were described as counterions of the bases in the salts used as PBG. One article, for example, discloses salts of 2-(3-benzoylphenyl)-, 2-(xanthone-2-yl)-, and 2-(thioxanthone-2-yl)propionic or acetic acid as car-boxylate-functional chromophores for PBG, amongst others. These excel absorption maxima at short wavelengths below 400 nm and are thus suitable for curing the formulations via UV irradiation. Cations of the salts used are, amongst others, those of peralkylated guanidines and phosphazenes (Zivic et al., Angew. Chem. Int. Ed. 58 (31), 10410-10422 (2019)).

In addition, the suitability of triarylphosphines as nucleophilic catalysts for oxa-Michael additions was disclosed recently, where triphenylphosphine, mono- and tri-methoxylated triphenylphosphine were studied with regard to their catalytic effect on 16 different combinations of Michael donors and acceptors. The use of tris(4-meth-oxyphenyl)phosphine tended to result in the best conversions, however, in almost half of the experiments there was hardly any difference between the three catalysts (Fischer et al., Beilstein J. Org. Chem. 17, 1689-1697 (2021)).

Against this background, it was the object of the invention to prepare new carboxylate salts and to use them as photobase generators in photopolymerizable compositions.

### DISCLOSURE OF THE INVENTION

In a first aspect, the present invention achieves this object by providing a novel photopolymerization method, namely by using a triphenylphosphonium carboxylate salt of the following Formula (I) as a photobase generator in a photopolymerizable composition comprising at least one type of monomers for preparing photopolymers via curing the composition by irradiating with light of a suitable wavelength:

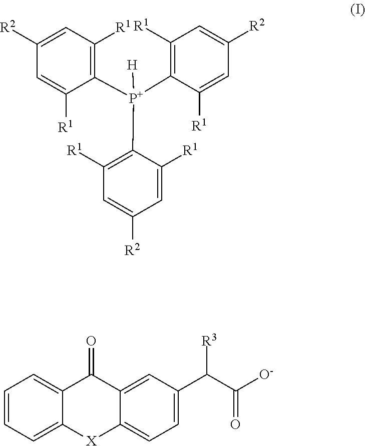

wherein R¹ and R² are each independently selected from —H and —OCH₃, wherein on each of the three phenyl radicals at least one of R¹ and R² represents —OCH₃, and wherein R³ is selected from —H and —CH₃ and X is either absent or selected from a chemical bond, —CH₂—, —O—CH₂—, —CH₂—O—, —C(═O)—, —O— or —S—.

The inventors have discovered that some salts consisting of such a triphenylphosphonium cation and the anion of one of the phenylacetic acid or phenylpropionic acid derivatives defined above, all of which were prepared for the first time by the inventors, are perfectly suited as photobase generators, as will be clearly demonstrated by the examples below. It was very surprising, though, that, on the contrary, some salts auf the same triphenylphosphonium cations, but with other anions, or of the same carbonate anion, but with other cations, proved to be completely unsuitable and resulted in no conversion of the reactants at all.

According to the invention, conversions were already achieved with mono-methoxylated phenyl radicals, wherein the methoxy group in the ortho position, i.e. with an —OCH₃ as R¹, led to somewhat better results than in the para position, i.e. with —OCH₃ as R². Much better results, however, were achieved with triphenylphosphonium cations with multi-methoxylated phenyl radicals. In preferred embodiments, at least one R¹ on each of the three phenyl radicals thus represents —OCH₃, more preferably both R¹ represent —OCH₃, and most preferably both R¹ and R² represent —OCH₃. This means that the phosphonium cation of the salt is preferably the tris(2-methoxy-phenyl)phosphonium cation, more preferably the tris(2,6-dimethoxyphenyl)phosphonium cation, most preferably the tris(2,4,6-trimethoxyphenyl)phosphonium cation.

Regarding the carboxylate anion, in preferred embodiments, X is absent or selected from a chemical bond, —O— or —S—. More preferably, it is the anion of one of the following carboxylic acids: 2-(3-benzoylphenyl)propionic acid (ketoprofen) or -acetic acid, 2-(xanthone-2-yl)propionic acid or -acetic acid or 2-(thioxanthone-2-yl)propionic acid or -acetic acid. In particular, the photobase generator is selected from the following triphenylphosphonium carboxylate salts: tris(2,6-dimethoxyphenyl)phosphonium-2-(3-benzoylphenyl)propionate, tris(2,4,6-trimethoxyphenyl)phosphonium-2-(3-benzoylphenyl)propionate, tris(2,6-dimethoxyphenyl)phosphonium-2-(xanthone-2-yl)acetate, tris(2,4,6-trimethoxyphenyl)phosphonium-2-(xanthone-2-yl)acetate, tris(2,6-dimethoxyphenyl)phosphonium-2-(thioxanthone-2-yl)acetate or tris(2,4,6-trimethoxyphenyl)phosphonium-2-(thioxanthone-2-yl)acetate, which led to excellent results.

The type of curing of the photopolymerizable composition is not particularly limited. In preferred embodiments, however, the photopolymerizable composition is either applied to a substrate and cured into a coating by irradiation, or cured into a three-dimensional object in a generative manufacturing method via layer-by-layer irradiation. In both cases, curing can be carried out under heating, and the photopolymer obtained thereby can be subjected to a thermal post-treatment to optimize the mechanical properties.

A preferred generative manufacturing method is hot lithography under heating to a temperature of at least 50° C. or at least 70° C. or at least 80° C., which leads to high conversions in very short times.

The inventive use is not limited to polymerization based on Michael additions, however, in preferred embodiments, at least two types of monomers polymerizable by Michael addition reactions are used in the photopolymerizable composition, which are, more preferably, polymerizable by oxa-ene additions, oxa-yne additions, or C—C addition reactions of C—H-active compounds, most preferably by oxa-ene additions. Particularly preferred examples of monomers are combinations of (meth)acrylates or (meth)acrylamides and alcohols, which are, especially with regard to generative manufacturing methods, preferably bulk reacted without solvent in the presence of the photobase generator.

In addition, the photopolymerizable compositions can also comprise at least one photosensitizer, preferably 9,10-dibutoxyanthracene, and/or at least one radical scavenger, preferably 2,6-di-tert-butyl-p-cresol (butylhydroxytoluene, BHT), in order to improve the reaction rate or conversion and to increase the storage stability of the composition.

In a second aspect, the present invention also provides a photopolymer obtainable by curing a photopolymerizable composition as described above.

In a third aspect, the present invention, of course, also provides the novel salts that were prepared by the inventors for the first time and are useable as photobase generators in the inventive method, i.e. a triphenylphosphonium carboxylate salt of the following Formula (I)

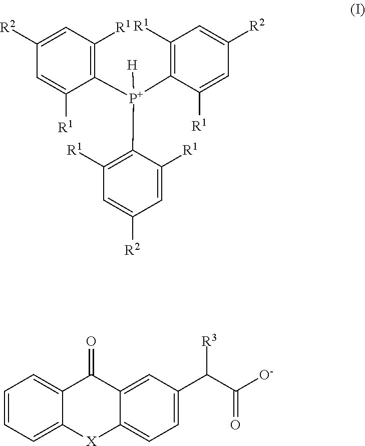

wherein R¹ and R² are each independently selected from —H and —OCH₃, and wherein R³ is selected from —H or —CH₃ and X is either absent or selected from —O— or —S—, wherein the salt is selected from the group consisting of the following:

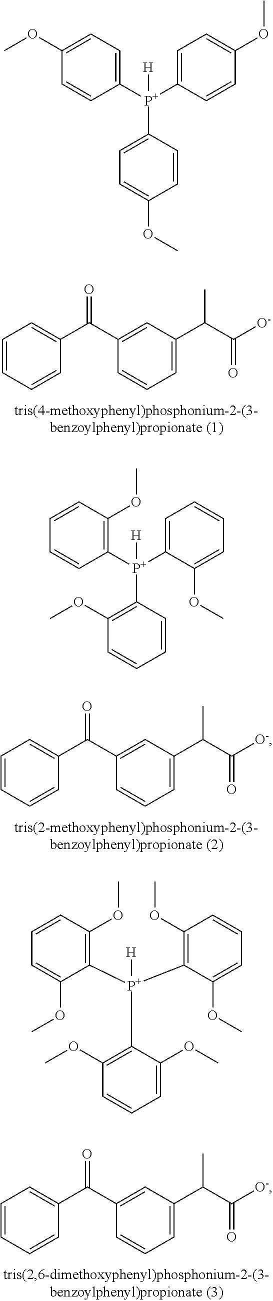

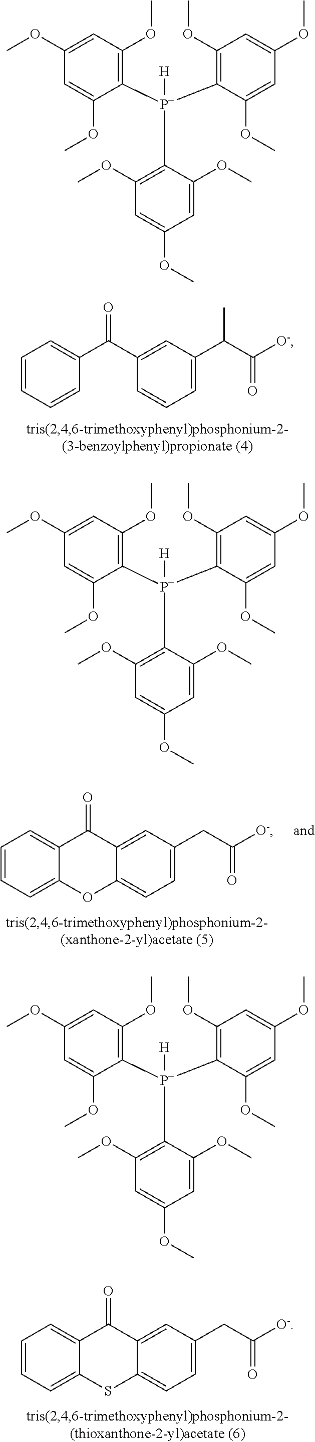

Finally, the invention also relates to the use of one of these new triphenylphosphonium carboxylate salts as a photobase generator in photopolymerizable compositions for preparing photopolymers.

### SHORT DESCRIPTION OF THE DRAWINGS

Below, the present invention will be described in more detail with reference to specific examples and the attached drawings which show the following:

FIG. 1 is a graphical representation of the reaction conversions achieved by varying the reaction temperature in Example 14.

FIG. 2 is a photo-DSC graph of the photopolymerization carried out in Example 16.

And FIGS. 3A-C, FIGS. 4A-B, FIGS. 5A-B, and FIGS. 6A-B are photographs of the three-dimensional bodies prepared in Example 22 by means of hot lithography.

### EXAMPLES

### Synthetic Examples—Examples 1 to 6, Comparative Examples 1 to 5

For preparing the novel phosphonium carboxylate salts, mainly commercial reagents, i.e. the respective triphenylphosphine (or in Comparative Example 2 BINAP, i.e. 2,2′-bis(diphenylphosphino)-1,1′-binaphthyl) and the corresponding carboxylic acid (or in Comparative Example 3 triethylammonium tetraphenylborate), were purchased and dried under high vacuum and used without further purification for forming salts. For Comparative Example 3, Example 5, and Example 6, the acid supplying the respective anion (i.e. triethylammonium tetraphenylborate, 9-oxo-9H-xanthene-2-acetic acid or 9-oxo-9H-thioxanthene-2-acetic acid) were prepared according to the literature, purified, and dried.

Salt formation was in each case carried out by providing a solution or suspension of the acid in abs. THF, adding an equimolar amount of the base (or, for Comparative Example 2, half the molar amount of the bisphosphine, BINAP), stirring for one hour, removing the solvent under high vacuum, washing the residue with an absolute solvent, and drying the salt under high vacuum. Most of the inventive examples resulted in quantitative conversions (>99% of th., “quant.”). Characterization was, in each case, carried out by NMR spectroscopy.

### Comparative Example 1 (CE1)

Unsubstituted triphenylphosphine and 2-(3-benzoylphenyl)propionic acid yielded triphenylphosphonium-2-(3-benzoylphenyl)propionate (CE1):

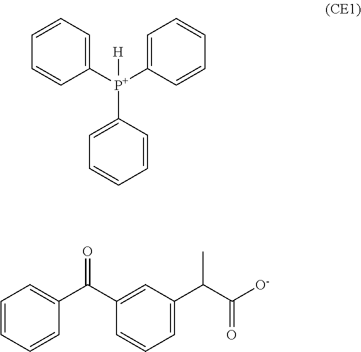 [SMILES: c1ccc([PH+](c2ccccc2)c2ccccc2)cc1 ; CC(C(=O)[O-])c1cccc(C(=O)c2ccccc2)c1]

2-(3-Benzoylphenyl)propionic acid (1 eq., 2 mmol, 0.509 g) and triphenylphosphine (1 eq., 2 mmol, 0.525 g) in abs. THF (6 ml) resulted, after removal of the solvent, in a sticky solid residue, which was washed with petroleum ether and dried, giving a white solid (yield: 0.896 g, 87% of th.).

¹H NMR (600 MHz, C₆D₆) δ: 7.85 (t, 1H), 7.70 (dd, 2H), 7.54 (dt, 1H), 7.47-7.33 (m, 6H), 7.23 (dt, 1H), 7.14-7.09 (m, 1H), 7.08-7.00 (m, 11H), 6.98 (t, 1H), 3.40 (q, 1H), 1.22 (d, 3H).

¹³C NMR (151 MHz, C₆D₆) δ: 195.31, 180.22, 140.21, 138.26, 137.73, 137.63, 133.91, 133.78, 131.96, 131.07, 129.99, 129.42, 128.98, 128.54, 128.51, 128.46, 128.42, 128.08, 45.04, 17.74.

³¹P NMR (243 MHz, C₆D₆) δ: −5.41.

### Example 1

Tris(4-methoxyphenyl)phosphine and 2-(3-benzoylphenyl)propionic acid yielded tris(4-methoxyphenyl)phosphonium-2-(3-benzoylphenyl)propionate (1):

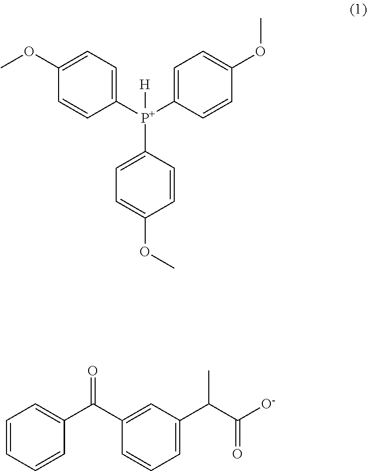

2-(3-Benzoylphenyl)propionic acid (1 eq., 1.8 mmol, 0.458 g) and tris(4-methoxy-phenyl)phosphine (1 eq., 1.8 mmol, 0.634 g) in abs. THF (7 ml) resulted, after removal of the solvent, in a white solid residue, which was washed with THF and dried, giving a white solid (yield: 1.092 g, quant.).

¹H NMR (600 MHz, C₆D₆) δ: 7.85 (s, 1H), 7.70 (dd, 2H), 7.53 (dt, 1H), 7.41 (dd, 5H), 7.23 (dt, 1H), 7.13-7.07 (m, 1H), 7.05-7.00 (m, 4H), 6.96 (t, 1H), 6.76-6.71 (m, 5H), 3.38 (q, 1H), 3.23 (s, 9H), 1.21 (d, 3H).

¹³C NMR (151 MHz, C₆D₆) δ: 194.42, 178.94, 159.48, 139.46, 137.39, 136.89, 134.40, 134.26, 131.08, 130.21, 129.11, 128.75, 128.58, 128.09, 127.55, 127.19, 113.43, 113.38, 53.49, 44.17, 16.93.

³¹P NMR (243 MHz, C₆D₆) δ: −10.32.

### Example 2

Tris(2-methoxyphenyl)phosphine and 2-(3-benzoylphenyl)propionic acid yielded tris(2-methoxyphenyl)phosphonium-2-(3-benzoylphenyl)propionate (2):

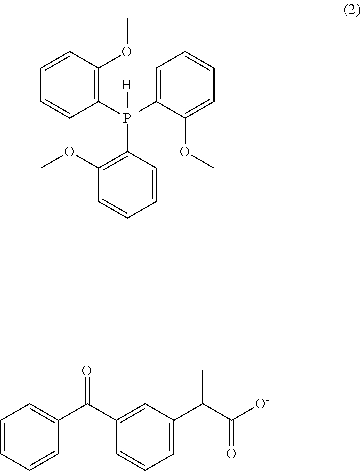 [SMILES: COc1ccccc1[PH+](c1ccccc1OC)c1ccccc1OC ; CC(C(=O)[O-])c1cccc(C(=O)c2ccccc2)c1]

2-(3-Benzoylphenyl)propionic acid (1 eq., 1.8 mmol, 0.458 g) and tris(2-methoxy-phenyl)phosphine (1 eq., 1.8 mmol, 0.634 g) in abs. THF (8 ml) resulted in, after removal of the solvent, a sticky solid residue, which was washed with diethyl ether and dried, giving a white solid (yield: 0.900 g, 82% of th.).

¹H NMR (600 MHz, C₆D₆) δ: 7.85 (s, 1H), 7.72-7.67 (m, 2H), 7.53 (dt, 2H), 7.23 (dt, 1H), 7.12-7.07 (m, 6H), 7.06-7.00 (m, 2H), 6.97 (t, 1H), 6.76 (td, 3H), 6.51 (m, 3H), 3.39 (q, 1H), 3.17 (s, 9H), 1.21 (d, 3H).

¹³C NMR (151 MHz, C₆D₆) δ: 195.29, 179.91, 161.85, 161.73, 140.28, 138.26, 137.74, 134.05, 131.96, 131.08, 129.95, 129.77, 128.96, 128.42, 128.08, 125.54, 125.43, 120.94, 110.06, 54.76, 45.03, 17.77.

### Example 3

Tris(2,6-dimethoxyphenyl)phosphine and 2-(3-Benzoylphenyl)propionic acid yielded tris(2,6-dimethoxyphenyl)phosphonium-2-(3-benzoylphenyl)propionate (3):

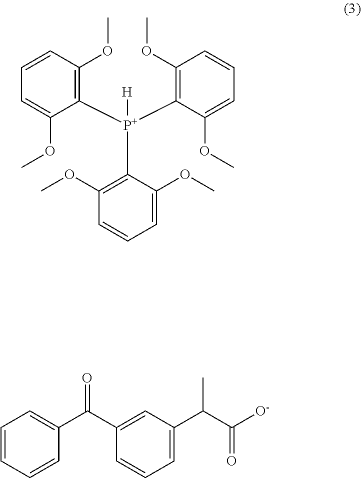 [SMILES: COc1cccc(OC)c1[PH+](c1c(OC)cccc1OC)c1c(OC)cccc1OC ; CC(C(=O)[O-])c1cccc(C(=O)c2ccccc2)c1]

2-(3-Benzoylphenyl)propionic acid (1 eq., 1.5 mmol, 0.381 g) and tris(2,6-dimethox-yphenyl)phosphine (1 eq., 1.5 mmol, 0.664 g) in abs. THF (7 ml) resulted, after removal of the solvent, in a white solid residue, which was washed with THF and dried, giving a white solid (yield: 1.045 g, quant.).

¹H NMR (600 MHz, C₆D₆) δ: 7.92 (d, 1H), 7.74-7.70 (m, 2H), 7.55 (dt, 1H), 7.38 (dt, 1H), 7.15-7.10 (tt, 3H), 7.03 (m, 3H), 7.10-7.02 (t, 1H), 6.31 (dd, 6H), 3.58 (q, 1H), 3.22 (s, 18H), 1.32 (d, 3H).

¹³C NMR (151 MHz, C₆D₆) δ: 195.44, 178.09, 162.73, 162.67, 141.32, 138.10, 137.84, 131.89, 131.41, 129.98, 129.49, 128.64, 128.59, 128.33, 128.03, 127.97, 104.35, 55.42, 45.40, 18.25.

### Example 4

Tris(2,4,6-trimethoxyphenyl)phosphine and 2-(3-benzoylphenyl)propionic acid yielded tris(2,4,6-trimethoxyphenyl)phosphonium-2-(3-benzoylphenyl)propionate (4):

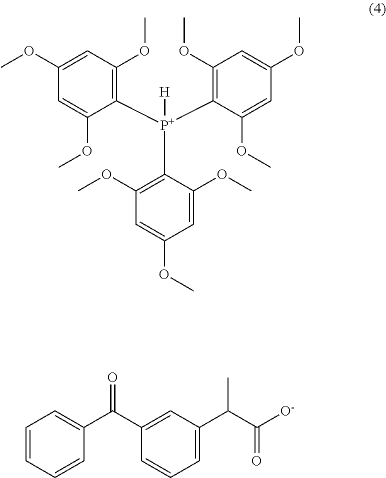

2-(3-Benzoylphenyl)propionic acid (1 eq., 2.38 mmol, 0.605 g) and tris(2,4,6-tri-methoxyphenyl)phosphine (1 eq., 2.38 mmol, 1.266 g) in abs. THF (9 ml) resulted in, after removal of the solvent, a beige, viscous solid residue, which was washed with diethyl ether and dried, giving a slightly beige solid (yield: 1.252 g, 67% of th.)

¹H NMR (600 MHz, C₆D₆) δ: 8.05 (t, 1H), 7.79-7.74 (m, 2H), 7.64 (dt, 1H), 7.57 (dt, 1H), 7.16-7.10 (m, 3H), 7.10-7.03 (m, 3H), 6.11 (d, 6H), 3.88 (q, 1H), 3.42 (s, 9H), 3.28 (s, 18H), 1.48 (d, 3H).

¹³C NMR (151 MHz, C₆D₆) δ: 195.82, 177.51, 163.72, 163.67, 143.51, 138.07, 137.69, 132.09, 131.73, 130.07, 129.71, 129.20, 128.10, 127.94, 91.49, 55.60, 55.48, 55.23, 54.62, 46.70, 19.12.

³¹P NMR (243 MHz, C₆D₆): −64.88.

### Comparative Example 2

2,2′-Bis(diphenylphosphino)-1,1′-binaphthyl and 2-(3-benzoylphenyl)propionic acid yielded 2,2′-bis(diphenylphosphonium)-1,1′-binaphthyl-bis[2-(3-benzoylphenyl)-propionate] (CE2):

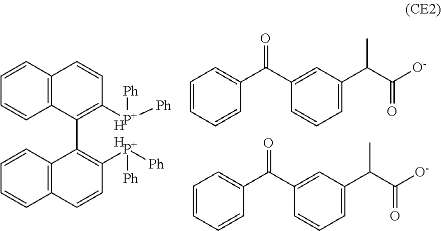

2-(3-Benzoylphenyl)propionic acid (2 eq., 1.6 mmol, 0.4068 g) and 2,2′-bis(diphenyl-phosphino)-1,1′-binaphthyl) (1 eq., 0.8 mmol, 0.498 g) in abs. THF (5 ml) resulted, after removal of the solvent, in a white solid residue, which was washed with THF and dried, giving a white solid (yield: 0.690 g, 76% of th.)

¹H NMR (400 MHz, C₆D₆) δ: 7.95 (t, 2H), 7.89-7.70 (m, 7H), 7.70-7.43 (m, 11H), 7.41-6.93 (m, 25H), 6.78 (m, 2H), 3.48 (q, 2H), 1.31 (d, 6H).

¹³C NMR (101 MHz, C₆D₆) δ: 195.24, 180.07, 140.20, 138.29, 137.75, 134.56, 134.34, 133.08, 132.98, 132.88, 131.92, 131.03, 129.94, 129.39, 128.96, 128.39, 128.06, 126.35, 125.72, 45.01, 17.18.

³¹P NMR (162 MHz, C₆D₆) δ: −15.04.

### Comparative Example 3

Tris(2,4,6-trimethoxyphenyl)phosphine and triethylammonium tetraphenylborate yielded tris(2,4,6-trimethoxyphenyl)phosphonium tetraphenylborate (CE3):

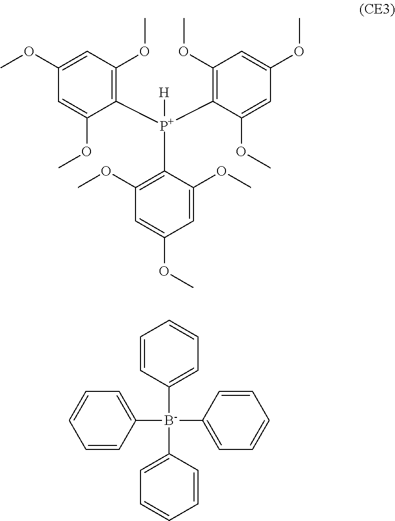

Triethylammonium tetraphenylborate (1 eq., 1.0 mmol, 0.421 g) prepared according to the literature (Faulkner et al., J. Am. Chem. Soc. 137 (22), 7224-7230 (2015)) and tris(2,4,6-trimethoxyphenyl)phosphine (1 eq., 1.0 mmol, 0.535 g) in abs. THF (7 ml) resulted, after removal of the solvent, in a white solid residue, which was washed with hexane and dried, giving a white solid (yield: 0.464 g, 54% of th.).

¹H NMR (600 MHz, acetone-d₆) δ: 7.35 (m, 8H), 6.94 (t, 8H), 6.79 (tt, 4H), 6.38 (d, 6H), 3.91 (s, 9H), 3.77 (s, 18H).

¹³C NMR (151 MHz, C₆D₆) δ: 205.32, 164.26, 136.16, 125.09, 121.36, 91.45, 91.40, 56.11, 55.40.

³¹P NMR (243 MHz, acetone-d₆) δ: −52.03.

### Comparative Example 4

Tris(2,4,6-trimethoxyphenyl)phosphine and phenylglyoxylic acid yielded tris(2,4,6-trimethoxyphenyl)phosphonium phenylglyoxylate (CE4):

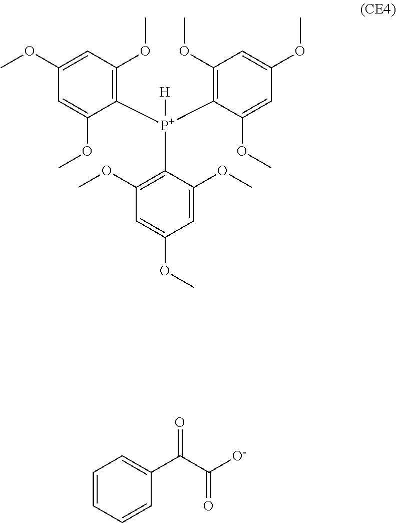

Phenylglyoxalic acid (1 eq., 1.0 mmol, 0.150 g) and tris(2,4,6-trimethoxyphenyl)phosphine (1 eq., 1.0 mmol, 0.533 g) in abs. THF (3 ml) resulted in, after removal of the solvent, a beige, sticky solid residue, which was washed with THF and dried, giving a slightly beige, sticky solid (yield: 0.683 g, quant.).

¹H NMR (400 MHz, acetone-d₆) δ: 7.90-7.83 (m, 2H), 7.46-7.38 (m, 1H), 7.32 (tt, 2H), 6.15 (d, 2H), 3.71 (d, 9H), 3.53 (s, 18H).

¹³C NMR (101 MHz, acetone-d₆) δ: 170.08, 163.19, 132.52, 129.39, 128.19, 91.35, 91.30, 55.79, 55.41, 55.08.

### Comparative Example 5

Tris(2,4,6-trimethoxyphenyl)phosphine and phthalimidoacetic acid yielded tris(2,4,6-trimethoxyphenyl)phosphonium phthalimidoacetate (CE5):

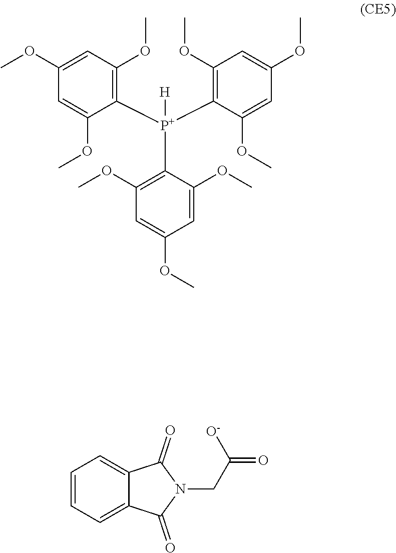

N-Phthaloglycine (1 eq., 1.0 mmol, 0.205 g) and tris(2,4,6-trimethoxyphenyl)phosphine (1 eq., 1.0 mmol, 0.533 g) in abs. THF (3 ml) resulted in, after removal of the solvent, a beige solid residue, which was washed with THF and dried, giving a slightly beige solid (yield: 0.738 g, quant.).

¹H NMR (400 MHz, acetone-d₆) δ: 7.89-7.81 (m, 4H), 6.25-6.14 (m, 6H), 4.34 (s, 2H), 3.83 (s, 9H), 3.59 (s, 18H).

¹³C NMR (101 MHz, acetone-d₆) δ: 168.74, 167.56, 163.54, 163.47, 133.99, 122.83, 91.18, 91.16, 55.46, 54.73, 40.96.

### Example 5

Tris(2,4,6-trimethoxyphenyl)phosphine and 2-(xanthone-2-yl) acetic acid yielded tris(2,4,6-trimethoxyphenyl)phosphonium-2-(xanthone-2-yl)acetate (5):

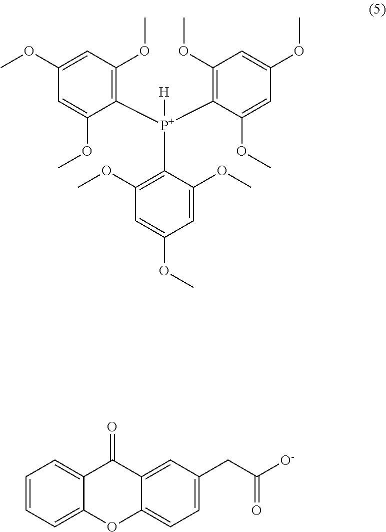

9—Oxo-9H-xanthene-2-acetic acid (1 eq., 0.2 mmol, 51 mg) prepared according to the literature (Blake et al., Org Lett. 8 (6), 1057-1060 (2006); with the exception that K₂CO₃ was used instead of Cs₂CO₃) and tris(2,4,6-trimethoxyphenyl)phosphine (1 eq., 0.2 mmol, 107 mg) in abs. THF (0.6 ml) resulted in, after removal of the solvent, a beige solid residue, which was washed with THF and dried, giving a slightly beige solid (yield: 158 mg, quant.).

¹H NMR (400 MHz, acetone-d₆) δ: 8.13 (dd, 1H), 8.06 (d, 1H), 7.77-7.66 (m, 2H), 7.51-7.40 (m, 2H), 7.38-7.29 (m, 1H), 6.03 (s, 6H), 3.66 (s, 9H), 3.61 (s, 2H), 3.39 (s, 18H).

¹³C NMR (101 MHz, acetone-d₆) δ: 171.64, 163.39, 136.66, 135.07, 126.60, 126.21, 124.05, 118.10, 117.98, 91.11, 55.32, 54.59, 39.67.

### Example 6

Tris(2,4,6-trimethoxyphenyl)phosphine and 2-(thioxanthone-2-yl) acetic acid yielded tris(2,4,6-trimethoxyphenyl)phosphonium-2-(thioxanthone-2-yl)acetate (6):

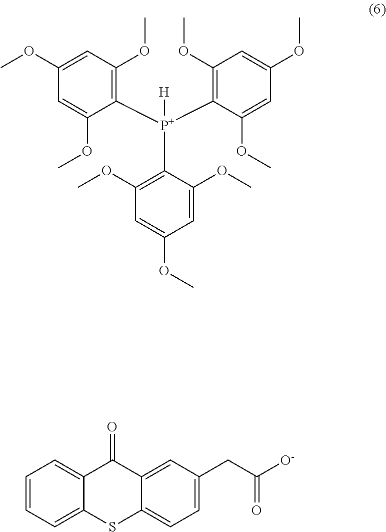

9—Oxo-9H-thioxanthene-2-acetic acid (1 eq., 0.15 mmol, 41 mg) prepared according to the literature (Yilmaz et al., Macromol. Rapid Commun. 37 (13), 1046-1051 (2016)) and tris(2,4,6-trimethoxyphenyl)phosphine (1 eq., 0.15 mmol, 80 mg) in abs. THF (0.5 ml) resulted in, after removal of the solvent, a light green solid residue, which was washed with THF and dried, giving a pale green solid (yield: 121 mg, quant.).

¹H NMR (400 MHz, acetone-d₆) δ: 8.42-8.29 (m, 2H), 7.63-7.54 (m, 4H), 7.49-7.35 (m, 1H), 5.97 (d, 6H), 3.84 (s, 0.3H), 3.69 (s, 1.7H), 3.64 (s, 9H), 3.37 (s, 18H).

¹³C NMR (101 MHz, acetone-d₆) δ: 163.25, 134.28, 132.58, 129.95, 129.35, 126.43, 126.31, 126.19, 91.06, 55.22, 54.48, 40.28.

### Examples 7 to 12, Comparative Examples 6 to 10-Photocatalysis “oxa-ene”

The new inventive compounds (1) to (6) prepared as described above and those of Comparative Examples (CE1) to (CE5) were initially investigated for their suitability as photobase generators in a photoinitiated model reaction. The reaction process was, in each case, monitored by photo-DSC (model: DSC 204 F1 Phoenix by Netzsch), and the conversions obtained thereby were then determined by means of ¹H NMR spectra of the reaction mixtures dissolved in CDCl₃. The model reaction used was an oxa-Michael addition between an alcohol and an acrylate, more spe-cifically between benzyl alcohol as a Michael donor and benzyl acrylate as a Michael acceptor, according to the following reaction:

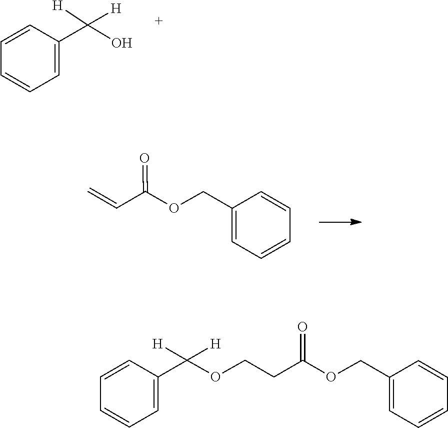 [SMILES: OCc1ccccc1 ; C=CC(=O)OCc1ccccc1 ; O=C(CCOCc1ccccc1)OCc1ccccc1]

For determining the conversion, the chemical shift of the two hydrogen atoms (high-lighted in the above scheme) of the methylene group of the benzyl alcohol was used. In the ¹H NMR spectra, they each resulted in a singlet, the peak of which appears at 4.63 ppm for the benzyl alcohol contained in the reaction mixtures as a reactant, after successful addition to the acrylate double bond, however, deep-field shifted to 4.43 ppm (while those for the methylene group of the benzyl alcohol bound as ester are all above 5 ppm).

The percentage of the conversion was consequently calculated according to the following formula:

$$U ( \% ) = {I}_{P} / ( {I}_{P} + {I}_{E} ) \times 100$$

wherein I_{P} represents the integral of the singlet peak in the product (i.e. at 4.43 ppm), and I_{E} represents that of the peak in the reactant (at 4.63 ppm).

The reaction mixtures of the model reactions were (expressed in molar amounts) in each case 1 eq. of benzyl alcohol, 1 eq. of benzyl acrylate, 2 mol % of the potential photobase generator, 0.02 mol % of 9,10-dibutoxyanthracene (BAnt) as photosensitizer and 2 mol % of 2,6-di-tert-butyl-p-cresol (butylhydroxytoluene, BHT) as a radical scavenger. 10 to 15 mg each of the formulations mixed at room temperature were filled into an aluminum DSC crucible and covered with a quartz glass panel. Subse-quently, the reaction mixtures were heated to 80° C., then irradiated at this temperature for 50 s with the medium pressure mercury lamp of the DSC device with light having a wavelength between 320 nm and 500 nm (with 133 mW/cm²), whereafter the temperature was maintained for another 850 s, followed by one repetition of 50 s of irradiation and 850 s maintaining (i.e. the reaction time was 2×15 min). Subse-quently, the reaction mixtures were cooled briefly and dissolved in CDCl₃, whereafter their ¹H NMR spectra were recorded.

The conversions calculated according to the above formula were as given in the following Table 1.

TABLE 1

| | | Conversion |
|---|---|---|
| Example | Compound | (%) |
| Comparative | (CE1) | 0 |
| Example 6 | | |
| Example 7 | (1) | 6 |
| Example 8 | (2) | 10 |
| Example 9 | (3) | 58 |
| Example 10 | (4) | 69 |
| Example 11 | (5) | 81 |
| Example 12 | (6) | 84 |
| Comparative | (CE2) | 0 |
| Example 7 | | |
| Comparative | (CE3) | 0 |
| Example 8 | | |
| Comparative | (CE4) | 0 |
| Example 9 | | |
| Comparative | (CE5) | 0 |
| Example 10 | | |

This clearly shows that none of the comparative substances was able to generate conversions, i.e. to be cleaved at irradiation and initiate the Michael addition reaction. This means that neither the unsubstituted triphenylphosphonium nor the BINAP cation in combination with inventive anions nor the inventive tris(2,4,6-trimethoxy-phenyl)phosphonium cation in combination with alternative anions are suitable as photobase generators.

With the inventive combinations of methoxy-substituted triphenylphosphonium cations and anions of phenylacetic acid or phenylpropionic acid derivatives it was, on the one hand, shown that the number of substituents as well as their positions have a strong impact on the reactivity: the ortho position of the methoxy group on the phenyl residues in Compound (2), i.e. as substituent R¹, is to be preferred over the para position in Compound (1), i.e. as substituent R². However, the conversions achieved therewith of 10% or only 6%, respectively, can be multiplied by increasing the number of substituents to two or even three: 58% conversion were obtained with the di-methoxy- and 69% with the trimethoxy-substituted Compound (3) or (4), respectively.

On the other hand, it is also shown that cyclization between the two benzol rings in the aromatic anion further improves the suitability of the salt as photobase generator: in combination with the tris(2,4,6-trimethoxyphenyl)phosphonium cation, the 2-(xan-thone-2-yl)acetate anion achieved 81% and the 2-(thioxanthone-2-yl)acetate anion 84% conversion for Compound (5) or (6), respectively. Therefore, the inventors assume that similar results will be achievable with a chemical bond, —CH₂—, —O—CH₂—, —CH₂—O—, or —C(═O)— as Substituent X in Formula (I)—and also with a longer alkyl radical as substituent R³ (such as ethyl or propyl). However, increasing the molecular weight of the photobase generator is, of course, not preferable as long it does not lead to significantly better results.

### Example 13, Comparative Example 11-Photocatalysis “C—C”

The new inventive Compound (4) and a known catalyst of the C—C Michael addition, namely CGI 1193 (by BASF) of the following Formula:

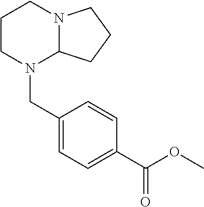 [SMILES: COC(=O)c1ccc(CN2CCCN3CCCC32)cc1]

were investigated in photo-DSC experiments analogous to those described above with regard to the conversions achievable in a model reaction with diethyl malonate as Michael donor and, again, benzyl acrylate as Michael acceptor according to the following reaction:

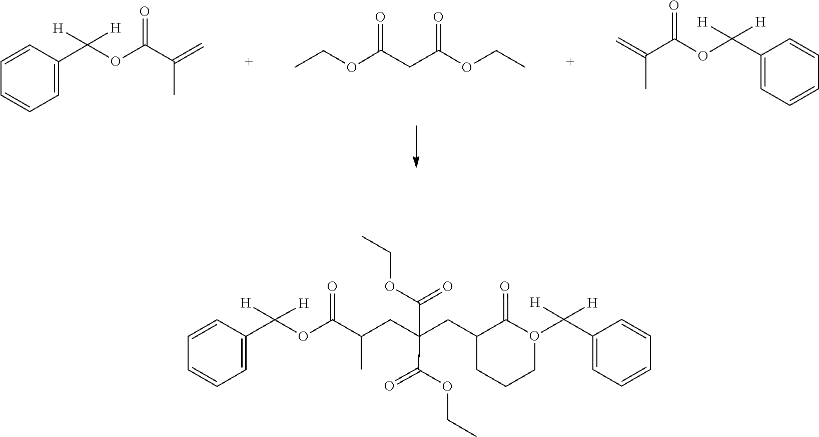

For determining the conversion, the chemical shifts of the two hydrogen atoms (high-lighted in the above scheme) of the benzyl methylene group of benzyl acrylate in the reactant and in the product were used.

Here, the reaction mixtures were in each case 1 eq. of diethyl malonate, 2 eq. of benzyl acrylate, 2 mol % of Compound (4) or CGI 1193, 0.02 mol % of BAnt as a photosensitizer and 2 mol % of BHT as a radical scavenger. The reaction conditions for Example 13 were the same as before (80° C., 2×50 s of irradiation, followed by 850 s of maintaining the temperature. The conversions calculated from the ¹H NMR spectra are shown in the following Table 2.

TABLE 2

| | | Conversion |
|---|---|---|
| Example | Compound | (%) |
| Comparative | CGI 1193 | 45 |
| Example 11 | | |
| Example 13 | (4) | 79 |

The novel inventive Compound (4) thus outperformed the known compound as the photobase generator with regard to the reaction conversion by 75%, which proves its excellent suitability also as a catalyst for C—C Michael additions.

### Example 14—Reaction Temperature

Since it was to be assumed that the conversions could be raised even more by increasing the concentration of the photobase generator or the sensitizer or the reaction temperature, respectively, the inventors conducted further photo-DSC experiments with Compound (4), with which a conversion of 69% had been achieved in the previous oxa-ene experiments.

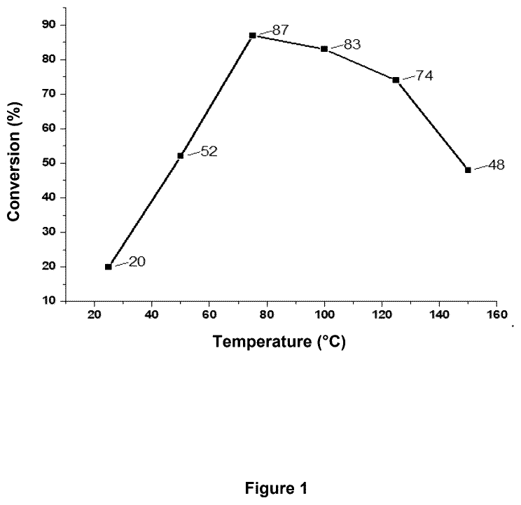

Here, the concentration of the photobase generator was increased from 2 to 5 mol %, and that of the sensitizer from 0.02 to 1 mol %, and the temperature of each mixture was increased starting from room temperature (25° C.) in steps of 25° C. each up to 150° C. The conversions, which were again calculated from the respective ¹H NMR spectra as described above, are shown in the following Table 3 and graphically de-picted in FIG. 1.

TABLE 3

| Temperature | Conversion |
|---|---|
| (° C.) | (%) |
| 25 | 20 |
| 50 | 52 |
| 75 | 87 |
| 100 | 83 |
| 125 | 74 |
| 150 | 48 |

On the one hand, these results suggest that the optimum reaction temperature for this reaction mixture should be in a range between 80° C. and 90° C. On the other hand, it has been shown that the reaction conversion could be increased significantly, i.e. by more than 25%, compared to the 69% achieved before with Compound (4) in Example 10, mainly by increasing the concentration of the photobase generator.

This further suggests that even the methoxy-monosubstituted Compounds (1) and (2), which led to comparatively poor results under the above reaction conditions in the Examples 7 and 8, could be used industrially as photobase generators after optimiz-ing the parameters. Of course, this also applies to ortho/para-disubstituted variants that have not been tested so far—in particular in combination with a ring-closed anion of Formula (I).

### Example 15—Storage Stability

To investigate the storage stability of the formulations, the model formulation investigated before in Example 10, which comprised 1 eq. of benzyl alcohol, 1 eq. of benzyl acrylate, 2 mol % of Compound (4), 2 mol % of BHT as a radical scavenger, and BAnt as a photosensitizer, was used again, wherein, in the present example, the amount of the sensitizer was increased from 0.02 mol % to 0.1 mol %, though. Therefrom, seve-ral mixtures were stored for up to 14 days at room temperature (RT) or at 60° C. under light exclusion, whereafter their compositions were again investigated by ¹H NMR, and any conversions were calculated as before. The results obtained are shown in the following Table 4.

TABLE 4

| Storage | | |
|---|---|---|
| duration | Conversion | Conversion |
| (days) | RT (%) | 60° C. (%) |
| 0 | 0 | 0 |
| 1 | 0 | 0 |
| 2 | 0 | 0 |
| 4 | 0 | 1 |
| 7 | 0 | 3 |
| 10 | 0 | 6 |
| 14 | 0 | 7 |

This clearly shows that 14 days at room temperature did not result in any reaction. At 60° C., first minor conversions were observed after 4 days, and even after 14 days, only 7% of the reactants had reacted at this increased temperature. Overall, this proves a more than satisfactory storage stability of the formulations according to the present invention.

### Example 16—Photopolymerization

To confirm the suitability of the inventive photobase generators for preparing photopolymers, a further photo-DSC experiment was conducted with the use of multifunc-tional monomers. In particular, a reaction mixture of 1 eq. of trimethylolpropane (TMP), 1 eq. of trimethylolpropane triacrylate (TMPTA), 5 mol % of Compound (4), 2 mol % of BHT as a radical scavenger, and BAnt as a photosensitizer was prepared, the amount of the latter was, however, again reduced to 0.02 mol %. The reaction temperature, however, was increased to 100° C.

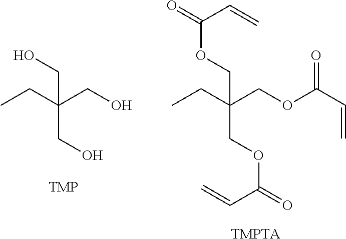

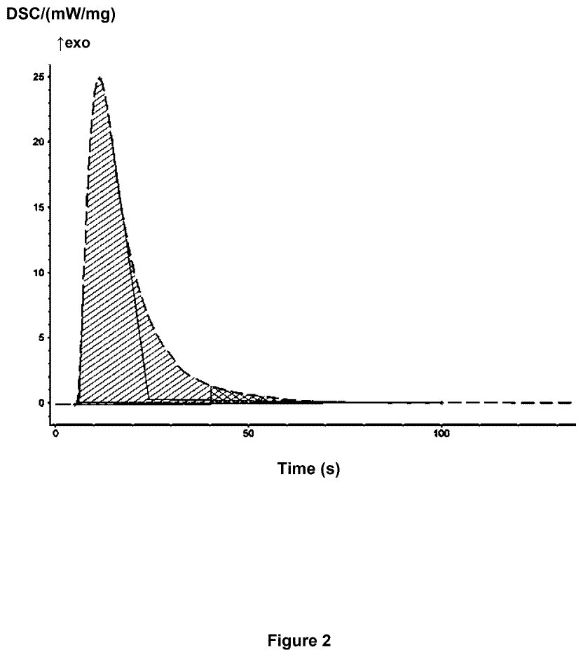

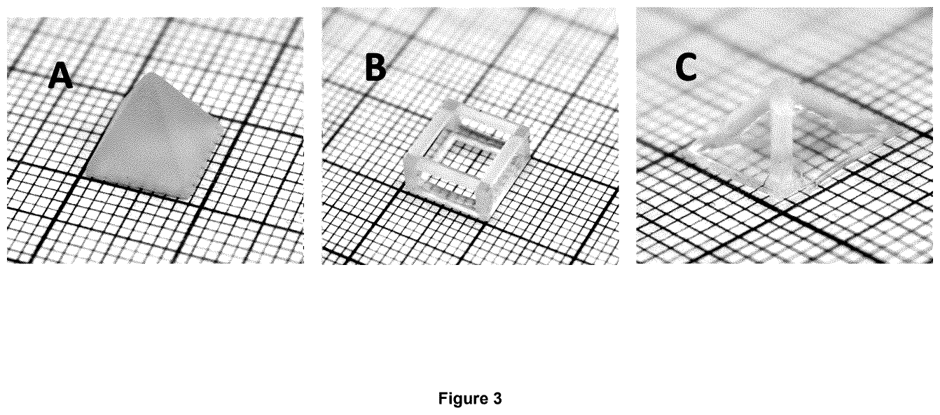

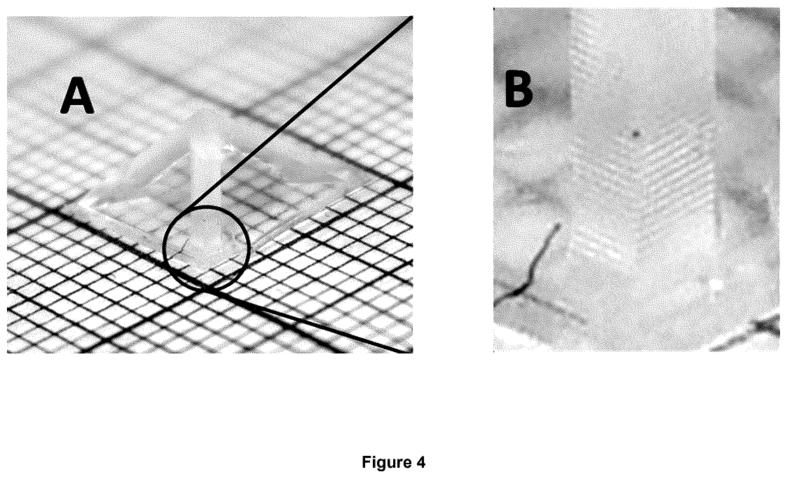

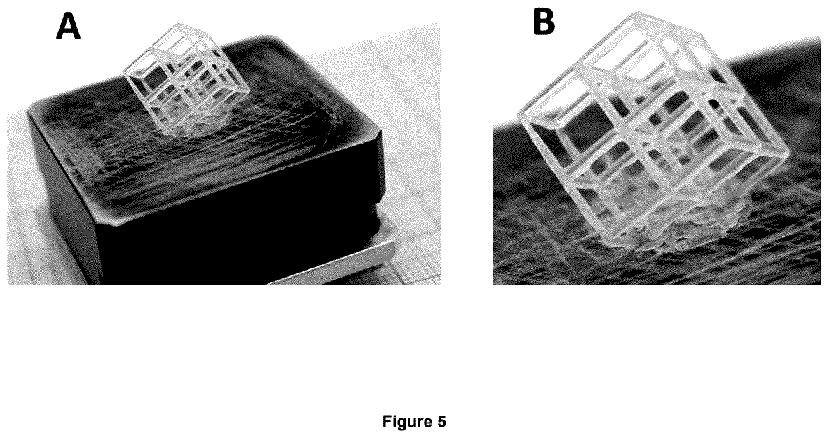

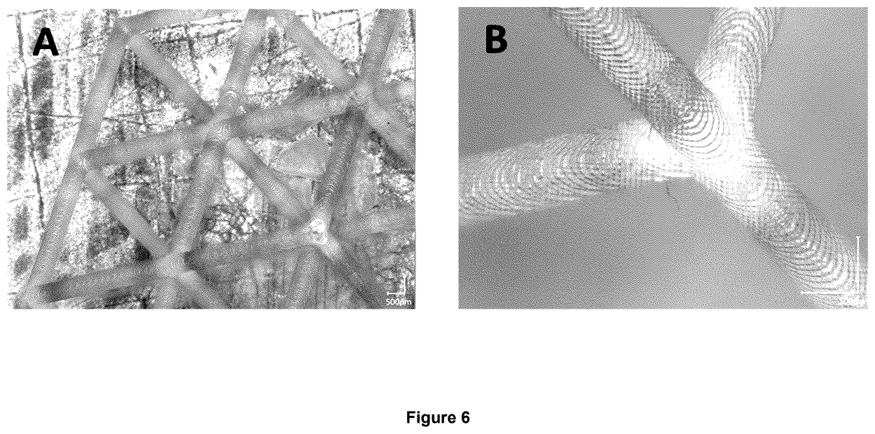

As can be seen in FIG. 2, strong exothermicity was observed only a few seconds after starting irradiation, which disappeared completely after just over one minute, which indicates the complete conversion of the reactants. Thus, the corresponding photopolymer could be prepared in a very short time with the inventive photobase generator (4).

### Example 17—Photopolymerization for Preparing a Coating

A reaction mixture of 1 eq. of TMP, 1 eq. of TMPTA, 2 mol % of Compound (4), 2 mol % of BHT as a radical scavenger, and 0.02 mol % of BAnt as a photosensitizer was applied to a glass panel using a doctor blade with a coating thickness of 100 μm.

This was heated to 80° C. in an INTELLI-RAY 600 UV oven by Uvitron International and irradiated with the mercury broadband UV lamp of the oven with UV light of 320-500 nm. After an irradiation time of only 1 min, a hard transparent coating was obtained on the glass panel.

### Examples 18 bis 21, Comparative Examples 12 and 13

For a more detailed determination of the reaction parameters of the above photopolymerization reaction, further photo-DSC experiments were conducted using reaction mixtures analogous to Example 17 consisting of 1 eq. of TMP, 1 eq. of TMPTA, 2 mol % of the respective photobase generator, 2 mol % of BHT as a radical scavenger, and 0.02 mol % of BAnt as a photosensitizer.

The performance of the inventive Compounds (1) to (4), i.e. the tris(methoxyphenyl)-phosphonium-2-(3-benzoylphenyl)propionates substituted at different positions or with a different number of methoxy groups, and of Comparative Examples 3 and 4 with different anions, i.e. tris(2,4,6-trimethoxyphenyl)phosphonium tetraphenylborate (CE3) and tris(2,4,6-trimethoxyphenyl)phosphonium phenylglyoxylate (CE4), was investigated.

From the respective photo-DSC graphs, the peak area (J/g) was determined as a measure for the polymerization rate, and the time until reaching 95% of the conversion, t₉₅ (s), as a measure for the rate of the total reaction. The results are shown in the following Table 5.

TABLE 5

| | | Area | Height | t₉₅ |
|---|---|---|---|---|
| Example | Compound | (J/g) | (mW/mg) | (s) |
| Comparative | (CE3) | 233 | 10.3 | 48.2 |
| Example 12 | | | | |
| Comparative | (CE4) | 155 | 5.3 | 52.5 |
| Example 13 | | | | |
| Example 18 | (1) | 328 | 19.3 | 40.1 |
| Example 19 | (2) | 299 | 18.3 | 38.6 |
| Example 20 | (3) | 311 | 18.7 | 39.0 |
| Example 21 | (4) | 326 | 20.7 | 38.7 |

At first glance, it is obvious that the two comparative substances, which had not been able to generate any conversion in the above Comparative Examples 8 and 9 as catalysts of the oxa-ene Michael addition between benzyl alcohol and benzyl acrylate, now showed activity as photobase generators. However, they were significantly outperformed by the four inventive compounds in all respects. The latter resulted in approximately 1.5-2 times larger peak areas, an approximately 2 or 4 times larger peak height, and approximately 20% shorter times for t₉₅, which shows that they were able to generate significantly higher conversions in a shorter time.

Compared to the above results of the photo-DSC experiments for the reaction between benzyl alcohol and benzyl acrylate it was not only surprising that (CE3) and (CE4) now acted as photobase generators as well, but also that all four inventive compounds yielded comparable results in the present Examples 18 to 21: none of the three parameters showed differences of more than 10%, even though the conversions achieved in the earlier Examples 7 bis 10 had varied significantly (6%, 10%, 58%, and 69%, respectively).

While not wishing to be bound by theory, the inventors assume that both is attributable to the Trommsdorff effect or gel effect that leads to an increase of the reaction rate during polymerizations with increasing conversions because the dissipa-tion of the reaction heat becomes increasingly difficult due to the simultaneously increasing viscosity, so that the exothermal polymerization reaction leads to an increase of the temperature. Of course, this effect is mainly observed in bulk polymerizations, i.e. without the dissolution effect of a solvent.

In any case, these results also prove the superiority of the inventive combinations of Formula (I) of methoxy-substituted triphenylphosphonium cations and the anions of defined phenylacetic acid or phenylpropionic acid derivatives over similar other salts, the cation and/or anion of which do not correspond to the definition according to the present invention.

### Example 22—Hot Lithography

Due to the fact that relatively high reaction temperatures were required or preferred for the tested reaction mixtures, they are particularly well suited for 3D printing via hot lithography, i.e. layer-by-layer irradiation of liquid mixtures with a laser under heating to elevated temperatures, for preparing three-dimensional photopolymers with pre-defined forms and structures.

For this purpose, four experiments with the same reaction mixture as in Example 16, i.e. 1 eq. of TMP, 1 eq. of TMPTA, 2 mol % of Compound (4), 2 mol % of BHT, and 0.02 mol % of BAnt, were carried out. The first three were heated to 80° C. in a Cubicure Caligma 200@ Hot Lithography System and cured layer-by-layer with a 375 nm laser (60 mW) and a scan rate of 200 mm/s with a layer thickness of 50 μm to give three-dimensional bodies. Photographs of the shaped bodies thus obtained are shown in FIGS. 3 and 4.

First, a massive square-based pyramid (FIG. 3A) was prepared, followed by a hollow cube (FIG. 3B). Then, a hollow pyramid was prepared, which is shown in FIG. 3C and FIG. 4, wherein in FIG. 4A the part of one of the four struts is marked, which is shown in an enlarged view in FIG. 4B, showing that the layers cured one by one during 3D printing are even visible to the naked eye.

Finally, a similar system as the Cubicure Caligma 200®, namely the Blue Printer 10 comprising an Ikarus II-Light-Engine by IN-VISION with a wavelength of 385 nm and a pixel pitch of 50 μm and thus capable of DLP hot lithography, was used to print a more complex cube shape shown in FIG. 5A and FIG. 5B (somewhat enlarged) from the same reaction mixture.

Printing was again conducted at 80° C. with an irradiation time of 18 s and an intensity of 75 mW/cm². In this case, however, Digital Light Processing, in short DLP, was used, where the light is guided onto the reaction mixture to be cured by means of a projector or beamer with reflective micro-mirrors instead of the laser.

In FIGS. 6A and 6B, light microscope images of this complex hollow cube are shown in different enlargements, showing the high resolutions of this 3D printing process.

All four experiments—as well as the other examples—clearly prove the suitability of the inventive triphenylphosphonium carboxylate salts according to Formula (I) as photobase generators.

1. A method for preparing photopolymers comprising:
providing a photopolymerizable composition comprising at least one type of monomer and a triphenylphosphonium carboxylate salt having Formula (I) as a photobase generator; and curing the composition by irradiating with UV light: 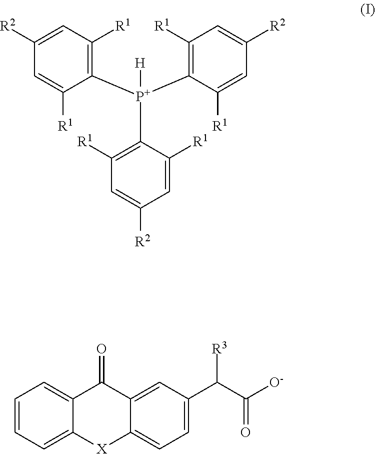
wherein R¹ and R² are each independently selected from —H and —OCH₃, wherein on each of the three phenyl radicals at least one of R¹ and R² represents —OCH₃, and wherein R³ is selected from —H and —CH₃ and X is either absent or selected from a chemical bond, —CH₂—, —O—CH₂—, —CH₂—O—, —C(═O)—, —O— or —S—.

2. The method according to claim 1, wherein at least one R¹ on each of the three phenyl radicals represents —OCH₃.

3. The method according to claim 2, wherein the phosphonium cation of the salt is the cation of one of the following triphenylphosphines: tris(2-methoxyphenyl)phosphine, tris(2,6-dimethoxyphenyl)phosphine or tris(2,4,6-trimethoxyphenyl)phosphine.

4. The method according to claim 1, wherein X is absent or selected form a chemical bond, —O— or —S—.

5. The method according to claim 4, wherein the carboxylate anion of the salt is the anion of one of the following carboxylic acids: 2-(3-benzoylphenyl)propionic acid or -acetic acid, 2-(xanthone-2-yl)propionic acid or -acetic acid or 2-(thioxanthone-2-yl)propionic acid or -acetic acid.

6. The method according to claim 5, wherein the photobase generator is selected from the following triphenylphosphonium carboxylate salts: tris(2,6-dimethoxyphenyl)phosphonium-2-(3-benzoylphenyl)propionate, tris(2,4,6-trimethoxyphenyl)phosphonium-2-(3-benzoylphenyl)propionate, tris(2,6-dimethoxyphenyl)-phosphonium-2-(xanthone-2-yl)acetate, tris(2,4,6-trimethoxyphenyl)phosphonium-2-(xanthone-2-yl)acetate, tris(2,6-dimethoxyphenyl)phosphonium-2-(thioxanthone-2-yl)acetate or tris(2,4,6-trimethoxyphenyl)phosphonium-2-(thioxanthone-2-yl)acetate.

7. The method according to claim 1, wherein the photopolymerizable composition is either applied to a substrate and cured into a coating by irradiation, or cured into a three-dimensional object in a generative manufacturing method via layer-by-layer irradiation, wherein curing is carried out under heating and wherein the photopolymer obtained thereby is optionally subjected to a thermal post-treatment.

8. The method according to claim 7, wherein hot lithography under heating to a temperature of at least 50° C. or at least 70° C. or at least 80° C. is conducted as said generative manufacturing method.

9. The method according to claim 1, wherein at least two types of monomers polymerizable by Michael addition reactions are used in the photopolymerizable composition.

10. The method according to claim 9, wherein the monomers are polymerizable by oxa-ene additions, oxa-yne additions or C—C addition reactions of C—H-active compounds.

11. The method according to claim 10, wherein a combination of (meth)acrylates or (meth)acrylamides is used, preferably in bulk without a solvent.

12. The method according to claim 1, wherein the photopolymerizable composition further comprises at least one photosensitizer and/or at least one radical scavenger.

13. A photopolymer obtained by curing the photopolymerizable composition by the method according to claim 1.

14. A triphenylphosphonium carboxylate salt of the following Formula (I) 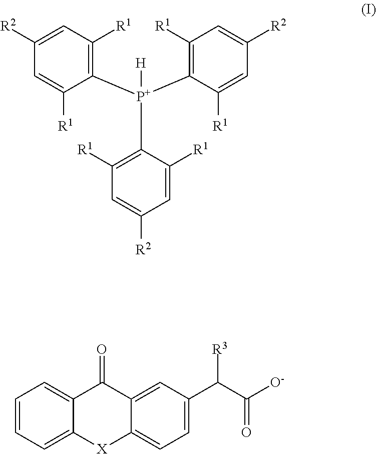
wherein R¹ and R² are each independently selected from —H or —OCH₃ and
wherein R³ is selected from —H or —CH₃ and X is either absent or selected from —O— or —S—, wherein the salt is selected from the group consisting of the following:

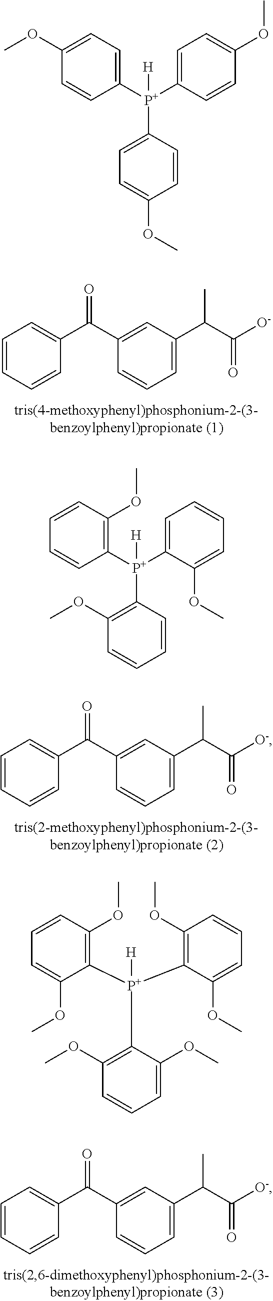

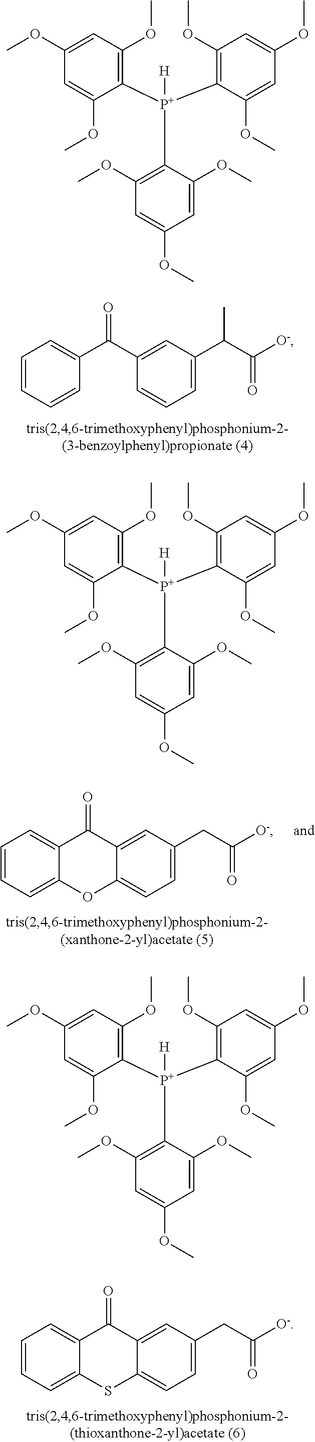

15. (canceled)
